(19) United States
(12) Patent Application Publication
(10) Pub. No.: US 2022/0415447 A1
(43) Pub. Date: Dec. 29, 2022

(54) METHOD FOR ASSESSING DRUG-RESISTANT MICROORGANISM AND DRUG-RESISTANT MICROORGANISM ASSESSING SYSTEM

(71) Applicant: China Medical University, Taichung City (TW)

(72) Inventors: Der-Yang Cho, Taichung City (TW); Jiaxin Yu, Taichung City (TW); Ni Tien, Taichung City (TW); Min-Hsuan Lu, Taichung City (TW); Chia-Fong Cho, Hemei Township (TW)

(73) Assignee: China Medical University, Taichung City (TW)

(21) Appl. No.: 17/850,165

(22) Filed: Jun. 27, 2022

(30) Foreign Application Priority Data
Jun. 29, 2021 (TW) 110123868

(51) Int. Cl. G16B 40/10; C12Q 1/02; G16B 40/20; H01J 49/40; H01J 49/16; H01J 49/00; G01N 27/64

(52) U.S. Cl. CPC G16B 40/10; C12Q 1/02; G16B 40/20; H01J 49/40; H01J 49/164; H01J 49/0036; G01N 27/64

(57) ABSTRACT

A method for assessing drug-resistant microorganism includes the following steps. A model establishing step is performed so as to obtain an antibiotic resistance assessing classifier. A test sample is provided. A sample pre-processing step is performed so as to obtain a processed sample. An analysis step is performed so as to obtain a target mass spectrum data. A spectrum pre-processing step is performed so as to obtain a normalized target mass spectrum data. A feature extraction step is performed so as to obtain a spectrum feature. An assessing step is performed, wherein the spectrum feature is analyzed by the antibiotic resistance assessing classifier so as to output an assessed result of drug-resistant microorganism, and the assessed result of drug-resistant microorganism is for assessing whether the test microorganism is a drug-resistant microorganism or not.

### RELATED APPLICATIONS

This application claims priority to Taiwan Application Serial Number 110123868, filed Jun. 29, 2021, which is herein incorporated by reference.

### BACKGROUND

### Technical Field

The present disclosure relates to a medical information analysis method and a system thereof. More particularly, the present disclosure relates to a method for assessing drug-resistant microorganism and a drug-resistant microorganism assessing system.

### Description of Related Art

Antimicrobial resistance is a naturally occurring phenomenon, but the abuse and misuse of antibiotics will accelerate the occurrence of the resistance to antibiotics of microorganisms. Furthermore, multidrug-resistant bacteria and pandrug-resistant bacteria (namely superbugs) have spread rapidly around the world, and it has become a common issue that is urgently addressed by related fields in the world.

Common bacterial infections, such as sepsis, meningitis, pneumonia, urinary tract infection, etc., are usually manifested as acute symptoms clinically. Thus, how to identify the species of microorganisms that causes the infection and the possible manifestation of antibiotics thereof is extremely important. In the current clinical, the gold standard to diagnose the bacterial infection is based on the microbial culture and the identification thereof as well as the results of antibiotic susceptibility testing in the laboratory, and then the administration of antibiotics is also based thereon. However, the current microbial culture and the identification thereof as well as the antibiotic susceptibility testing need to take at least 72 hours to issue a complete microbial culture report. Accordingly, not only there is a risk of delaying treatment, but the prognosis of the disease is not ideal.

In order to solve the aforementioned problems, the matrix-assisted laser desorption ionization-time of flight (MALDI-TOF) mass spectrometry is applied to identify the species of microorganism that causes the infection clinically so as to shorten the time for identifying the bacterial species based on biochemical methods. However, after the species of the microorganism is confirmed, the subsequent antibiotic susceptibility testing thereof still needs to take at least 24 hours. As a result, it is too late for the treatment of the acute symptoms caused by bacterial infection.

Therefore, how to provide a rapid and accurate method to identify the microorganisms causing bacterial infections and the results of antibiotic susceptibility testing thereof so as to provide a more reliable basis for the use of antibiotics has become the goal of the relevant academic and industry development.

### SUMMARY

According to one aspect of the present disclosure, a method for assessing drug-resistant microorganism includes the following steps. A model establishing step is performed, wherein the model establishing step includes the following steps: a drug-resistance database is provided, wherein the drug-resistance database includes a plurality of reference mass spectrum data, and the reference mass spectrum data are obtained by processing a processed reference sample with a conventional sample processing method or a rapid sample processing method; a reference spectrum pre-processing step is performed, wherein the reference mass spectrum data are pre-processed so as to obtain a plurality of normalized reference mass spectrum data; and a model training step is performed, wherein the normalized reference mass spectrum data are trained to achieve a convergence by an algorithm classifier so as to obtain an antibiotic resistance assessing classifier. A test sample is provided, wherein the test sample includes a test microorganism. A sample pre-processing step is performed, wherein the test sample is processed by the conventional sample processing method or the rapid sample processing method so as to obtain a processed sample. An analysis step is performed, wherein the processed sample is detected by a mass spectrometry method so as to obtain a target mass spectrum data. A spectrum pre-processing step is performed, wherein the target mass spectrum data is pre-processed so as to obtain a normalized target mass spectrum data. A feature extraction step is performed, wherein the normalized target mass spectrum data is trained to achieve a convergence by the antibiotic resistance assessing classifier so as to obtain a spectrum feature. An assessing step is performed, wherein the spectrum feature is analyzed by the antibiotic resistance assessing classifier so as to output an assessed result of drug-resistant microorganism, and the assessed result of drug-resistant microorganism is for assessing whether the test microorganism is a drug-resistant microorganism or not.

According to another aspect of the present disclosure, a drug-resistant microorganism assessing system includes a non-transitory machine readable medium and a processor. The non-transitory machine readable medium is for storing a target mass spectrum data, wherein the target mass spectrum data is obtained by detecting a processed sample by a mass spectrometry method, the processed sample includes a test microorganism, and the processed sample is obtained by a conventional sample processing method or a rapid sample processing method. The processor is signally connected to the non-transitory machine readable medium, wherein the processor includes a drug-resistant microorganism assessing program, and the drug-resistant microorganism assessing program includes a spectrum pre-processing module and an antibiotic resistance assessing classifier. The spectrum pre-processing module is for pre-processing the target mass spectrum data so as to obtain a normalized target mass spectrum data, wherein the spectrum pre-processing module includes a calibration unit, a sampling normalization unit and a spectrum conversion unit. The calibration unit is for removing a background noise of the target mass spectrum data so as to obtain a first processed target mass spectrum data. The sampling normalization unit is signally connected to the calibration unit, wherein the sampling normalization unit is for adjusting a temporal resolution value of the first processed target mass spectrum data so as to obtain a second processed target mass spectrum data. The spectrum conversion unit is signally connected to the sampling normalization unit, wherein the spectrum conversion unit is for processing the second processed target mass spectrum data by a mass-to-charge ratio conversing method so as to obtain a converted mass spectrum data, and then a data interval value of the converted mass spectrum data is adjusted by the spectrum conversion unit so as to obtain the normalized target mass spectrum data. The antibiotic resistance assessing classifier is signally connected to the spectrum pre-processing module, wherein the normalized target mass spectrum data is trained to achieve a convergence by the antibiotic resistance assessing classifier so as to obtain a spectrum feature, and the spectrum feature is analyzed by the antibiotic resistance assessing classifier so as to output an assessed result of drug-resistant microorganism. The antibiotic resistance assessing classifier is established by a model establishing step, wherein the model establishing step includes following steps. A drug-resistance database is provided, wherein the drug-resistance database includes a plurality of reference mass spectrum data, and the reference mass spectrum data are obtained by processing a processed reference sample with a conventional sample processing method or a rapid sample processing method. A reference spectrum pre-processing step is performed, wherein the reference mass spectrum data are pre-processed so as to obtain a plurality of normalized reference mass spectrum data. A model training step is performed, wherein the normalized reference mass spectrum data are trained to achieve a convergence by an algorithm classifier so as to obtain the antibiotic resistance assessing classifier. The assessed result of drug-resistant microorganism is for assessing whether the test microorganism is a drug-resistant microorganism or not.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a method for assessing drug-resistant microorganism according to one embodiment of the present disclosure.

FIG. 2 is a flow chart of Step 140 of the method for assessing drug-resistant microorganism of FIG. 1.

FIG. 3 is a block diagram of a drug-resistant microorganism assessing system according to another embodiment of the present disclosure.

FIG. 4 shows assessed results of drug-resistant microorganisms obtained by the antibiotic resistance assessing classifier of the present disclosure that is used to analyze the conventional mass spectrometry data of different drug resistance microorganisms.

FIG. 5A shows analyzed results of the sliding window algorithm of the methods for assessing drug-resistant microorganism of Example 1 to Example 5, wherein “100 Da” represents that the slide and the calculation are made in a frequency in an increment of 100 daltons every time.

FIG. 5B shows analyzed results of the sliding window algorithm of the methods for assessing drug-resistant microorganism of Example 6 to Example 10, wherein “100 Da” represents that the slide and the calculation are made in a frequency in an increment of 100 daltons every time.

FIG. 6A is a rapid mass spectrometry data of the sample infected with Acinetobacter baumannii.

FIG. 6B is a conventional mass spectrometry data of the sample infected with Acinetobacter baumannii.

FIG. 6C is a rapid mass spectrometry data of the sample infected with Staphylococcus aureus.

FIG. 6D is a conventional mass spectrometry data of the sample infected with Staphylococcus aureus.

### DETAILED DESCRIPTION

The present disclosure will be further exemplified by the following specific embodiments. However, the readers should understand that the present disclosure should not be limited to these practical details thereof, that is, in some embodiments, these practical details are used to describe how to implement the materials and methods of the present disclosure and are not necessary.

[Method for Assessing Drug-Resistant Microorganism of the Present Disclosure]

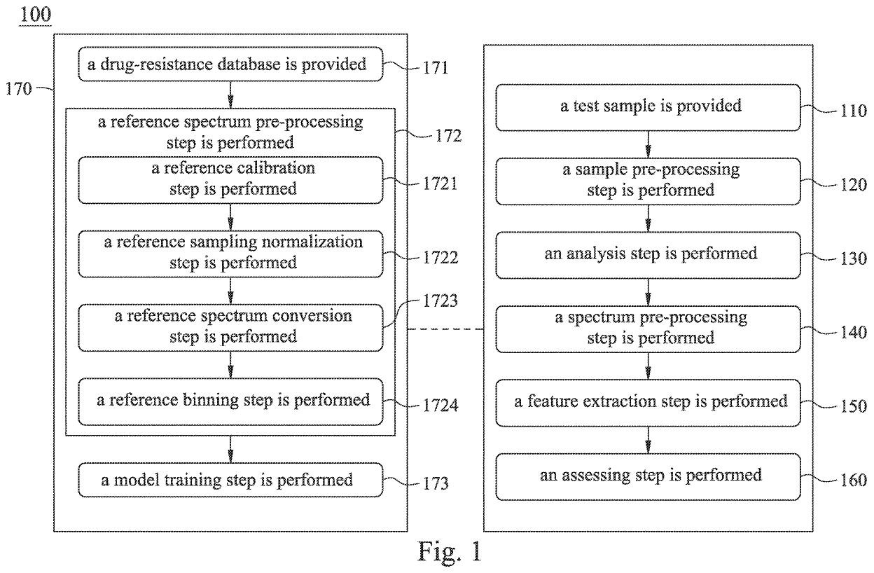

Please refer to FIG. 1, which shows a flow chart of a method 100 for assessing drug-resistant microorganism according to one embodiment of the present disclosure. The method 100 for assessing drug-resistant microorganism is for assessing whether a test microorganism is a drug-resistant microorganism or not, and the method 100 for assessing drug-resistant microorganism includes Step 110, Step 120, Step 130, Step 140, Step 150, Step 160 and Step 170.

In Step 110, a test sample is provided, wherein the test sample includes the test microorganism. The test sample can be blood, body fluids, tissues, excrement, excreta and other samples of patients suffering from bacterial infection, but the present disclosure is not limited thereto.

In Step 120, a sample pre-processing step is performed, wherein the test sample is processed by a conventional sample processing method or a rapid sample processing method so as to obtain a processed sample. In detail, in the current clinical procedures for diagnosing bacterial infections, the sample of the patient are cultured first so as to obtain microorganisms therein for species identification, and then the microorganisms are taken for incremental culture for antibiotic susceptibility testing or mass spectrometry analysis. However, the microbial culture and the identification thereof as well as the antibiotic susceptibility testing need to take at least 72 hours, and it is not favorable for treating the bacterial infections that progress rapidly and timely. On the contrary, in the method 100 for assessing drug-resistant microorganism of the present disclosure, the test sample not only can be processed by the conventional sample processing method but also can be processed by the rapid sample processing method, and both of the processed samples obtained therefrom can be used for the following analysis. Accordingly, the method 100 for assessing drug-resistant microorganism of the present disclosure not only is suitable for analyzing the processed sample obtained from the current clinical processing procedures, but also can be efficiently converted and analyzed under the premise that the test sample is processed by the rapid sample processing method. Therefore, the method 100 for assessing drug-resistant microorganism of the present disclosure can effectively reduce the time required for conventional microbial culture, identification and antibiotic susceptibility testing, so that the breadth of applications thereof is excellent.

Furthermore, the current clinical routine diagnostic method for species identification by Matrix Assisted Laser Desorption Ionization Time-of-Flight mass spectrometry (“MALDI-TOF” hereafter) mass spectrometry (referred to as the conventional sample processing method in the present disclosure) is described as follows. First, the bacterial samples are separated and grown on a solid agar medium. After incubation for 24 to 48 hours, a single colony is picked and smeared onto a spot on a MALDI-TOF target plate. Next, the sample spot is overlaid with 1 μL of 70% formic acid and dried at room temperature. Another 1 μL of a-cyano-4-hydroxycinnamic acid (CHCA) matrix solution is added to the sample spot. Once the sample spot has dried, the MALDI-TOF target plate applied to the MALDI-TOF mass spectrometer for analysis. However, the conventional sample processing method can be performed by any other method suitable for applying in the present disclosure, and it is not limited to the aforementioned description.

Furthermore, in the method 100 for assessing drug-resistant microorganism of the present disclosure, the test sample can be processed by a step-by-step centrifuging method in the rapid sample processing method, and the step-by-step centrifugation method includes the following steps. A centrifuging step is performed, wherein the test sample is processed by a plurality of centrifugations so as to obtain a centrifuged sample, and the centrifuged sample includes the test microorganism. A reactive step is performed, wherein a reaction reagent is added to the centrifuged sample and then well mixed so as to obtain a post-reaction sample. A final centrifuging step is performed, wherein the post-reaction sample is centrifuged so as to obtain the processed sample. The reaction reagent includes thioglycolate broth, ethanol, formic acid or acetonitrile. In detail, the step-by-step centrifuging method is obtained by adaptively adjusting the rapid microbial identification method mentioned in Table 1 of the journal published by Léa Ponderand et al. in 2020 as well as the rapid microbial identification method (C&W method) mentioned in the journal published by Ni Tien et al. in 2020. Through different centrifugation speeds and different centrifugation times, the step-by-step centrifuging method can gradually recover the test microorganisms from the positive blood culture fluid or peritoneal dialysate, and then the reaction reagent including ethanol, formic acid and acetonitrile (Léa Ponderand et al.), or the thioglycolate broth (Ni Tien et al.) is added to the centrifugated sample and fully reacted. After centrifugation again, the processed sample can be obtained.

Furthermore, in the method 100 for assessing drug-resistant microorganism of the present disclosure, the rapid sample processing method also can process the test sample by a commercial kit based on the user manual thereof and then for the analysis of the following mass spectrometry method. In detail, when the test sample is processed by the commercial kit, the lysis buffer of the commercial kit is added to the test sample and well mixed, and then is processed at different centrifugation speeds and different centrifugation times according to the instructions in the user manual so as to obtain a centrifuged sample including the test microorganism. Then, the rinse buffer or other reagents of the commercial kit are added therein and then centrifuged again, and then the processed sample can be obtained. Moreover, in the present disclosure, the commercial kit can be MBT Sepsityper® IVD kit, Vitek MS Blood culture Kit®, Rapid BACpro® II kit, Rapid BACpro® II kit, Rapid Sepsityper® protocol or Complete Sepsityper® protocol, but the present disclosure is not limited thereto.

In Step 130, an analysis step is performed, wherein the processed sample is detected by a mass spectrometry method so as to obtain a target mass spectrum data. In detail, the mass spectrometry method used in the present disclosure is MALDI-TOF method. In particular, in the application of the identification of the clinical microorganisms, different types (liquid or solid) of samples can be mixed with detection reagents (substrates) in the MALDI-TOF method. Next, the sample will be excited by a laser light to form gas-phase ions, and then the mass-to-electron ratios of gas-phase ions are detected by a mass spectrometer and then converted into a mass spectrum data. Finally, the mass spectrum data of the sample is compared with that of known microorganisms based on the consistency of the mass spectrum of the same species so as to finish the identification of the species of the microorganisms. Accordingly, by using the MALDI-TOF method to detect the processed sample, the method 100 for assessing drug-resistant microorganism of the present disclosure can be close to the current clinical process used to identify microorganisms, and it has not only a high market acceptance but also a high assessing accuracy in the related application.

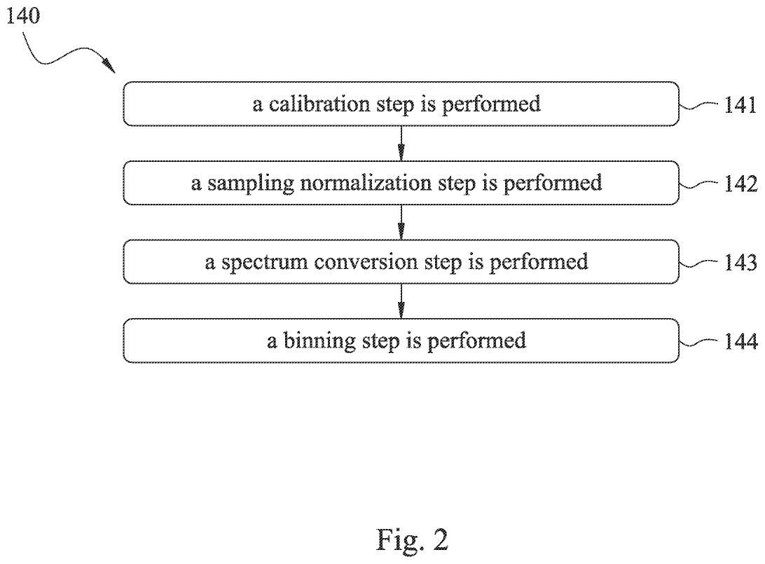

Please refer to FIG. 1 and FIG. 2 simultaneously, wherein FIG. 2 is a flow chart of Step 140 of the method 100 for assessing drug-resistant microorganism of FIG. 1. In Step 140, a spectrum pre-processing step is performed, and Step 140 includes Step 141, Step 142, Step 143 and Step 144.

In Step 141, a calibration step is performed, wherein a background noise of the target mass spectrum data is removed so as to obtain a first processed target mass spectrum data. In detail, before Step 141 is performed, the target mass spectrum data obtained by detecting the processed sample will be examined initially. If the target mass spectrum data includes blank portions or the format thereof does not match, the said mass spectrum data will be removed from the following analysis. Then, the signals of the target mass spectrum data will be smoothed so as to remove the background noise thereof.

In Step 142, a sampling normalization step is performed, wherein a temporal resolution value of the first processed target mass spectrum data is adjusted so as to obtain a second processed target mass spectrum data. In detail, in the sampling normalization step, the raw signal resolution and the sampling frequency of the first processed target mass spectrum data will be checked whether there is an inconsistency or not. If so, the first processed target mass spectrum data will be resampled so as to make the temporal resolution values thereof consistent. Then, a baseline correction will be performed by the top-hat method and the following Formula (I) is used to normalize the signal intensity so as to make the signal resolution consistent, so that the second processed target mass spectrum data can be obtained. Formula (I) is shown below:

z=(x−μ)/σ Formula (I);

wherein “z” represents z-score, “x” represents the mass-to-charge ratio intensity of each point of the first processed target mass spectrum data, “μ” represents the average signal intensity of the first processed target mass spectrum data, and “σ” represents the standard deviation of intensity of the first processed target mass spectrum data. After normalizing the intensity of the first processed target mass spectrum data, the mass-to-charge ratio intensity data with negative z-scores represents that subtle signals or noise are existed and will be further removed.

In Step 143, a spectrum conversion step is performed, wherein the second processed target mass spectrum data is processed by a mass-to-charge ratio conversing method so as to obtain a converted mass spectrum data. Preferably, a mass-to-charge ratio of the converted mass spectrum data can range from 2,000 to 20,000 daltons (Da).

In Step 144, a binning step is performed, wherein a data interval value of the converted mass spectrum data is adjusted so as to obtain the normalized target mass spectrum data. In detail, the peaks of the converted mass spectrum data are often shifted due to the effects of the isotopes in the mass spectrometry method. Thus, in order to prevent the peaks of the converted mass spectrum data from shifting, the method 100 for assessing drug-resistant microorganism of the present disclosure uses a proper data interval value to bin the converted mass spectrum data so as to obtain the normalized target mass spectrum data with a normalized data interval value. Preferably, the data interval value of the converted mass spectrum data can range from 1 to 10 daltons, and a mass-to-charge ratio of the normalized target mass spectrum data can range from 2,000 to 14,000 daltons. More preferably, the data interval value of the converted mass spectrum data can be 10 daltons, and the mass-to-charge ratio of the normalized target mass spectrum data can range from 4,000 to 12,000 daltons for the following analysis.

Furthermore, it must be noted that in the present disclosure, the terms “first” and “second” are only for naming and are not used to express quality or have other meanings.

In Step 150, a feature extraction step is performed, wherein the normalized target mass spectrum data is trained to achieve a convergence by the antibiotic resistance assessing classifier so as to obtain a spectrum feature. In detail, the time dimension data of the normalized target mass spectrum data will be analyzed automatically in the feature extraction step so as to output a corresponding spectrum feature. Thus, the spectrum feature can be obtained without manual or other methods, so that the use thereof is more convenient.

In Step 160, an assessing step is performed, wherein the spectrum feature is analyzed by the antibiotic resistance assessing classifier so as to output an assessed result of drug-resistant microorganism. Furthermore, in the method 100 for assessing drug-resistant microorganism of the present disclosure, the drug-resistant microorganism can be Methicillin-Resistant Staphylococcus aureus (MRSA), Vancomycin-Resistant Enterococci (VRE), Carbapenem-Resistant Acinetobacter baumannii (CRAB), Carbapenem-Resistant Pseudomonas aeruginosa (CRPA), Carbapenem-Resistant Klebsiella pneumoniae (CRKP), Carbapenem-Resistant Escherichia coli (CREC), Carbapenem-Resistant Escherichia cloacae (CRECL) or Carbapenem-Resistant Morganella morganii (CRMM), but the present disclosure is not limited thereto.

In Step 170, a model establishing step is performed, and Step 170 includes Step 171, Step 172 and Step 173.

In Step 171, a drug-resistance database is provided, wherein the drug-resistance database includes a plurality of reference mass spectrum data, and the reference mass spectrum data are obtained by processing a processed reference sample with a conventional sample processing method or a rapid sample processing method. Furthermore, the drug-resistance database can further include an antibiotic dataset, a drug susceptibility dataset and a microbial species dataset. The microbial species dataset includes names of bacteria species, Gram stain types thereof, colony patterns and other information, and each of the reference mass spectrum data can respectively correspond to a pathogenic microorganism information, a drug susceptibility report or an antibiotic information, but the present disclosure is not limited thereto. Furthermore, the reference mass spectrum data can be a MALDI-TOF mass spectrum data so as to be close to the current clinical process used to identify microorganisms.

In Step 172, a reference spectrum pre-processing step is performed, wherein the reference mass spectrum data are pre-processed so as to obtain a plurality of normalized reference mass spectrum data, and the reference spectrum pre-processing step includes Step 1721, Step 1722, Step 1723 and Step 1724.

In Step 1721, a reference calibration step is performed, wherein a background noise of each of the reference mass spectrum data is removed so as to obtain a plurality of first processed reference mass spectrum data. In detail, before Step 1721 is performed, each of the reference mass spectrum data will be examined initially. If one of the reference mass spectrum data includes blank portions or the format thereof does not match, the said reference mass spectrum data will not be used to establish the antibiotic resistance assessing classifier of the present disclosure. Then, the signals of each of the reference mass spectrum data will be smoothed so as to remove the background noise thereof.

In Step 1722, a reference sampling normalization step is performed, wherein a temporal resolution value of each of the first processed reference mass spectrum data is adjusted so as to obtain a plurality of second processed reference mass spectrum data. In detail, in the reference sampling normalization step, the raw signal resolution and the sampling frequency of all of the reference mass spectrum data will be respectively checked whether there is an inconsistency or not. If so, all the reference mass spectrum data will be resampled so as to make the temporal resolution values thereof consistent. Furthermore, the baseline of each of the reference mass spectrum data will be corrected by the top-hat method and then normalized according to the aforementioned Formula (I) so as to normalize the signal intensity, so that the signal resolution of all the reference mass spectrum data can be consistent. Furthermore, the details of Formula (I) are shown in the aforementioned paragraph and will not be described herein again.

In Step 1723, a reference spectrum conversion step is performed, wherein each of the second processed reference mass spectrum data is processed by a mass-to-charge ratio conversing method so as to obtain a plurality of converted reference mass spectrum data. Preferably, a mass-to-charge ratio of each of the converted reference mass spectrum data can range from 2,000 to 20,000 daltons.

In Step 1724, a reference binning step is performed, wherein a reference data interval value of each of the converted reference mass spectrum data is adjusted so as to obtain the normalized reference mass spectrum data. In detail, in order to prevent the peaks of each of converted reference mass spectrum data or the peaks between different converted reference mass spectrum data from shifting, a proper reference data interval value will be used to bin each of the converted reference mass spectrum data in the reference binning step so as to obtain the plurality of normalized reference mass spectrum data with the same reference data interval value. Preferably, the reference data interval value of each of the converted reference mass spectrum can range from 1 to 10 daltons, and a mass-to-charge ratio of each of the normalized reference mass spectrum data can range from 2,000 to 14,000 daltons. More preferably, the reference data interval value of each of the converted reference mass spectrum can be 10 daltons, and the mass-to-charge ratio of each of the normalized reference mass spectrum data can range from 4,000 to 12,000 daltons for the following analysis.

In Step 173, a model training step is performed, wherein the normalized reference mass spectrum data are trained to achieve a convergence by an algorithm classifier so as to obtain the antibiotic resistance assessing classifier. Preferably, the algorithm classifier can be a boosting algorithm classifier. Furthermore, the algorithm classifier can be LightGBM (Light Gradient Boosting Machine) algorithm classifier, CatBoost algorithm classifier, XGBoost (Extreme Gradient Boosting) algorithm classifier, Gradient Boosting algorithm classifier or other algorithm classifiers based on the decision tree algorithm, but the present disclosure is not limited thereto.

Therefore, by the procedure that the processed sample is obtained according to the conventional sample processing method or the rapid sample processing method, and then the target mass spectrum data corresponding to processed sample is processed sequentially by the calibration step, the sampling normalization step, the spectrum conversion step and the binning step and then trained to achieve the convergence by the antibiotic resistance assessing classifier so as to output the assessed result of drug-resistant microorganism, the method 100 for assessing drug-resistant microorganism of the present disclosure can effectively reduce the time required for conventional microbial culture, identification and antibiotic susceptibility testing. Further, a more reliable test result for the subsequent clinical use of antibiotics also can be provided correspondingly, so that the method 100 for assessing drug-resistant microorganism of the present disclosure has the potential application in relevant markets.

[Drug-Resistant Microorganism Assessing System of the Present Disclosure]

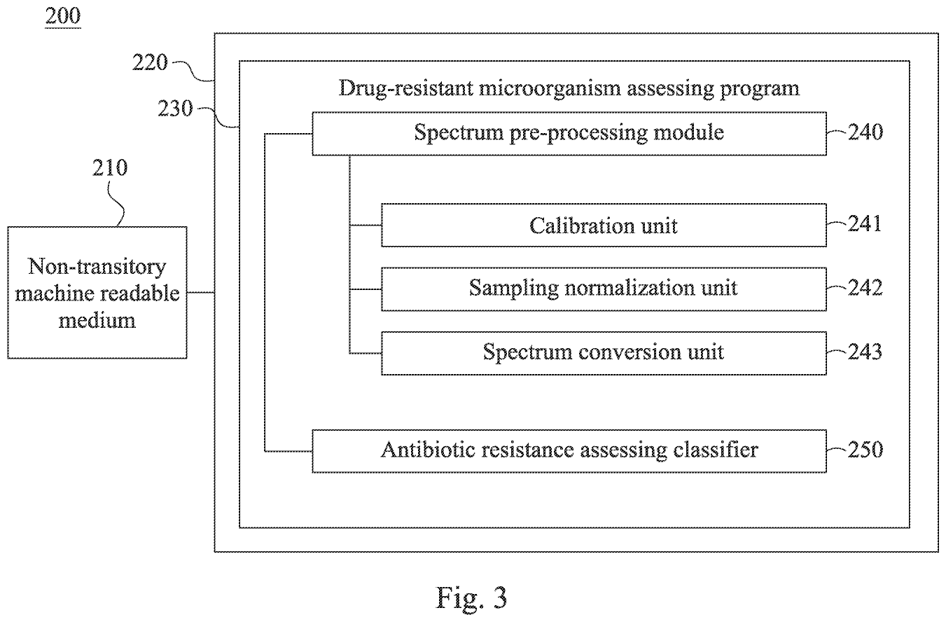

Please refer to FIG. 3, which is a block diagram of a drug-resistant microorganism assessing system 200 according to another embodiment of the present disclosure. The drug-resistant microorganism assessing system 200 is for assessing whether the test microorganism is a drug-resistant microorganism or not, and the drug-resistant microorganism assessing system 200 includes a non-transitory machine readable medium 210 and a processor 220.

The non-transitory machine readable medium 210 is for storing a target mass spectrum data, wherein the target mass spectrum data is obtained by detecting a processed sample by a mass spectrometry method so as to obtain a target mass spectrum data. In detail, the non-transitory machine readable medium can be electronically or signally connected to a mass spectrometer (not shown), wherein the mass spectrometer is for detecting the processed sample so as to obtain the target mass spectrum data, and then the target mass spectrum data will be output by the mass spectrometer to the non-transitory machine readable medium. Further, the processed sample includes a test microorganism, and the processed sample is obtained by a conventional sample processing method or a rapid sample processing method. The rapid sample processing method can be performed by a commercial kit (such as MBT Sepsityper® IVD kit) based on the user manual thereof so as to obtain the processed sample, and the mass spectrometry method can be the MALDI-TOF method. Furthermore, the drug-resistant microorganism can be Methicillin-Resistant Staphylococcus aureus (MRSA), Vancomycin-Resistant Enterococci (VRE), Carbapenem-Resistant Acinetobacter baumannii (CRAB), Carbapenem-Resistant Pseudomonas aeruginosa (CRPA), Carbapenem-Resistant Klebsiella pneumoniae (CRKP), Carbapenem-Resistant Escherichia coli (CREC), Carbapenem-Resistant Escherichia cloacae (CRECL) or Carbapenem-Resistant Morganella morganii (CRMM), but the present disclosure is not limited thereto.

The processor 220 is signally connected to the non-transitory machine readable medium 210, wherein the processor 220 includes a drug-resistant microorganism assessing program 230, and the drug-resistant microorganism assessing program 230 includes a spectrum pre-processing module 240 and an antibiotic resistance assessing classifier 250.

The spectrum pre-processing module 240 is for pre-processing the target mass spectrum data so as to obtain a normalized target mass spectrum data, wherein the spectrum pre-processing module 240 includes a calibration unit 241, a sampling normalization unit 242 and a spectrum conversion unit 243.

The calibration unit 241 is for removing a background noise of the target mass spectrum data so as to obtain a first processed target mass spectrum data.

The sampling normalization unit 242 is signally connected to the calibration unit 241, wherein the sampling normalization unit 242 is for adjusting a temporal resolution value of the first processed target mass spectrum data so as to obtain a second processed target mass spectrum data.

The spectrum conversion unit 243 is signally connected to the sampling normalization unit 242, wherein the spectrum conversion unit 243 is for processing the second processed target mass spectrum data by a mass-to-charge ratio conversing method so as to obtain a converted mass spectrum data, and then a data interval value of the converted mass spectrum data is adjusted by the spectrum conversion unit 243 so as to obtain the normalized target mass spectrum data. Preferably, the data interval value of the converted mass spectrum data can range from 1 to 10 daltons, and a mass-to-charge ratio of the converted mass spectrum data can range from 2,000 to 20,000 daltons. More preferably, the data interval value of the converted mass spectrum data can be 10 daltons, and the mass-to-charge ratio of the converted mass spectrum data can range from 4,000 to 12,000 daltons.

Further, the connecting relationship of the calibration unit 241, the sampling normalization unit 242 and the spectrum conversion unit 243 can be adjusted according to actual needs, and the present disclosure is not limited to FIG. 3 and the description thereof.

Furthermore, because the operation details of the calibration unit 241, the sampling normalization unit 242 and the spectrum conversion unit 243 are similar with the details described in Step 1721, Step 1722, Step 1723, Step 1724 of FIG. 1 and Step 141, Step 142, Step 143, Step 144 of FIG. 2, so that the details thereof will not be described herein again.

The antibiotic resistance assessing classifier 250 is signally connected to the spectrum pre-processing module 240, wherein the normalized target mass spectrum data is trained to achieve a convergence by the antibiotic resistance assessing classifier 250 so as to obtain a spectrum feature, and the spectrum feature is analyzed by the antibiotic resistance assessing classifier 250 so as to output an assessed result of drug-resistant microorganism. Furthermore, the antibiotic resistance assessing classifier 250 is established by a model establishing step, and the model establishing step is the same to Step 170 of the method 100 for assessing drug-resistant microorganism of FIG. 1, so that the details thereof will not be described herein again.

Furthermore, in the drug-resistant microorganism assessing system 200 of the present disclosure, the non-transitory machine readable medium 210 and the processor 220 can respectively be a physical system or a cloud system. In detail, if the non-transitory machine readable medium 210 is a physical system, it can be electronically connected to the mass spectrometer and signally connected to the processor 220 so as to capture the target mass spectrum data from the mass spectrometer directly, and then the target mass spectrum data will be sent to the processor 220, which is a physical system or a cloud system, for further analysis. Conversely, if the non-transitory machine readable medium 210 is a cloud system, the non-transitory machine readable medium 210 will be signally connected to the processor 220, and the target mass spectrum data will be stored on the Internet through a cloud computing provider who manages and operates data storage as a service. However, the present disclosure is not limited thereto.

Moreover, the drug-resistant microorganism assessing system 200 of the present disclosure also can further include a display interface (not shown) for displaying the assessed result of drug-resistant microorganism, but the present disclosure is not limited thereto.

Therefore, by the arrangements of the calibration unit 241, the sampling normalization unit 242 and the spectrum conversion unit 243 of the spectrum pre-processing module 240 as well as the antibiotic resistance assessing classifier 250, the assessed result of drug-resistant microorganism output by the drug-resistant microorganism assessing system 200 of the present disclosure can be used to assess whether the test microorganism is the drug-resistant microorganism or not effectively and accurately. Furthermore, the drug-resistant microorganism assessing system 200 of the present disclosure can effectively reduce the time required for the conventional microbial culture, the identification and the antibiotic susceptibility testing, and then the harm caused by the complications of microbial infections can be reduced. Thus, the breadth of applications the drug-resistant microorganism assessing system 200 of the present disclosure is excellent.

### Example

I. Drug-Resistance Database

The drug-resistance database of the present disclosure is for establishing the antibiotic resistance assessing classifier of the present disclosure. In detail, the reference mass spectrum data of the drug-resistance database are mass spectrometry data of samples collected by China Medical University Hospital, and the aforementioned clinical research study is approved by China Medical University & Hospital Research Ethics Committee, which are numbered as CMUH109-REC3-098. The mass spectrometry data are obtained after the samples are processed by the conventional sample processing method in the clinical laboratory, and the drug-resistance database includes the mass spectrometry data of the samples infected by bacteria of Methicillin-Resistant Staphylococcus aureus (“MRSA” hereafter), Vancomycin-Resistant Enterococci (“VRE” hereafter), Carbapenem-Resistant Acinetobacter baumannii (“CRAB” hereafter), Carbapenem-Resistant Pseudomonas aeruginosa (“CRPA” hereafter), Carbapenem-Resistant Klebsiella pneumoniae (“CRKP” hereafter), Carbapenem-Resistant Escherichia coli (“CREC” hereafter), Carbapenem-Resistant Escherichia cloacae (“CRECL” hereafter) and Carbapenem-Resistant Morganella morganii (“CRMM” hereafter). The numbers of the samples of the drug-resistant bacteria are listed in Table 1.

TABLE 1

| | | | | | | | | |
|---|---|---|---|---|---|---|---|---|
| Drug-resistant | MRSA | VRE | CRAB | CRPA | CRKP | CREC | CRECL | CRMM |
| bacteria | | | | | | | | |
| Sample | 16 | 10 | 8 | 15 | 18 | 44 | 4 | 2 |
| number | | | | | | | | |
| (thousand) | | | | | | | | |

Simultaneously, each of the reference mass spectrum data corresponds to a pathogenic microorganism information, a drug susceptibility report or an antibiotic information of the test sample, and the aforementioned information will be imported into the drug-resistance database in JSON (JavaScript Object Notation) format and then for the establishment of the antibiotic resistance assessing classifier.

II. Reference Spectrum Pre-Processing

In the present test, the raw signals of the reference mass spectrum data of the drug-resistance database are processed by Python (version 3.6), which is used as the processing method of the spectrum pre-processing module of the present disclosure.

First, each of the reference mass spectrum data will be examined initially by the calibration unit of the spectrum pre-processing module so as to confirm the raw signal condition thereof, and then the reference mass spectrum data which has blank portions or the format thereof does not match will be removed. Then, the signals of each of the reference mass spectrum data will be smoothed by the calibration unit of the spectrum pre-processing module so as to remove the background noise thereof and obtain a plurality of first processed reference mass spectrum data.

Next, the raw signal resolution and the sampling frequency of the first processed reference mass spectrum data will be checked by the sampling normalization unit of the spectrum pre-processing module so as to make sure that there is an inconsistency or not. If so, all the reference mass spectrum data will be resampled so as to make the temporal resolution values thereof consistent. Furthermore, the baseline of each of the reference mass spectrum data will be corrected by the top-hat method and then normalized according to the aforementioned Formula (I) so as to normalize the signal intensity. Thus, the signal resolution of all the reference mass spectrum data can be consistent and a plurality of second processed reference mass spectrum data can be obtained.

Finally, the second processed target mass spectrum data after calibrating and sampling normalizing will be processed by a mass-to-charge ratio conversing method by the spectrum conversion unit of the spectrum pre-processing module so as to obtain a plurality of converted reference mass spectrum data. Then, the data interval value will be respectively adjusted based on 1 dalton, 5 daltons, 10 daltons, 15 daltons and 20 daltons so as to estimate the best range of the data interval value of the converted reference mass spectrum and then prevent the peaks of each of converted reference mass spectrum data or the peaks between different converted reference mass spectrum data from shifting. Then, a plurality of normalized reference mass spectrum data can be obtained for establishing the antibiotic resistance assessing classifier of the present disclosure.

After reference spectrum pre-processing is finished, each of the reference mass spectrum data will be further labeled with the pathogenic microorganism information, the drug susceptibility report or the antibiotic information correspondingly as the basis for the subsequent analysis of the drug-resistant microorganism assessment.

III. Reliability Analysis of the Method for Assessing Drug-Resistant Microorganism and the Drug-Resistant Microorganism Assessing System of the Present Disclosure

1. Analyzing the Assessing Accuracy of the Antibiotic Resistance Assessing Classifier of the Present Disclosure

In the present test, the MALDI-TOF mass spectrum data of MRSA in the drug-resistance database are trained by different boosting algorithm classifiers and normalized in different ways, and then the assessing accuracies of the antibiotic resistance assessing classifier obtained therefrom are analyzed.

The boosting algorithm classifiers used in the present test include LightGBM algorithm classifier, CatBoost algorithm classifier, XGBoost algorithm classifier and Gradient Boosting algorithm classifier. Simultaneously, other types of algorithm classifiers are used to train the MALDI-TOF mass spectrum data of MRSA in the drug-resistance database so as to further illustrate the assessing accuracy of the antibiotic resistance assessing classifier of the present disclosure. The other types of algorithm classifiers described above include Extra Trees algorithm classifier, Logistic Regression algorithm classifier, Random Forest algorithm classifier, Ada Boost algorithm classifier, Decision Tree algorithm classifier, Linear Discriminant Analysis algorithm classifier, K Neighbors algorithm classifier, Naive Bayes algorithm classifier and Quadratic Discriminant Analysis algorithm classifier.

Please refer to Table 2, Table 3 and Table 4, wherein Table 2 shows the data that the reference mass spectrum data are processed by the z-score normalization and then trained with different algorithm classifiers, Table 3 shows the data that the reference mass spectrum data are processed by the Min-Max normalization and then trained with different algorithm classifiers, and Table 4 shows the data that the reference mass spectrum data are processed by the z-score normalization and the Min-Max normalization and then trained with different algorithm classifiers.

TABLE 2

| | | | | | | Kappa |
|---|---|---|---|---|---|---|
| Algorithm | | | | | | coeffi- |
| classifier | Accuracy | AUC | Recall | Precision | F1-sore | cient |
| CatBoost | 0.8134 | 0.8981 | 0.7909 | 0.8367 | 0.8131 | 0.6272 |
| Light GBM | 0.8108 | 0.8928 | 0.7919 | 0.8315 | 0.8111 | 0.6219 |
| XGBoosting | 0.7872 | 0.8757 | 0.7504 | 0.8199 | 0.7834 | 0.5751 |
| Gradient | 0.787 | 0.8728 | 0.7510 | 0.8192 | 0.7834 | 0.5747 |
| Boosting | | | | | | |
| Extra Trees | 0.7849 | 0.8712 | 0.7374 | 0.8250 | 0.7787 | 0.5708 |
| Logistic | 0.7744 | 0.8294 | 0.7698 | 0.7862 | 0.7779 | 0.5488 |
| Regression | | | | | | |
| Random | 0.7445 | 0.8264 | 0.6815 | 0.7917 | 0.7324 | 0.4905 |
| Forest | | | | | | |
| Ada Boost | 0.7440 | 0.822 | 0.7299 | 0.7613 | 0.7451 | 0.4882 |
| Decision | 0.6951 | 0.6963 | 0.7135 | 0.6988 | 0.7060 | 0.3895 |
| Tree | | | | | | |
| Linear | 0.6584 | 0.6785 | 0.6743 | 0.6650 | 0.6694 | 0.3160 |
| Discriminant | | | | | | |
| Analysis | | | | | | |
| K Neighbors | 0.6213 | 0.6691 | 0.6518 | 0.6258 | 0.6384 | 0.2412 |
| Naive | 0.5341 | 0.5792 | 0.4385 | 0.6048 | 0.4015 | 0.0730 |
| Bayes | | | | | | |
| Quadratic | 0.5207 | 0.5306 | 0.1525 | 0.6818 | 0.2297 | 0.0600 |
| Discriminant | | | | | | |
| Analysis | | | | | | |

TABLE 3

| | | | | | | Kappa |
|---|---|---|---|---|---|---|
| Algorithm | | | | | | coeffi- |
| classifier | Accuracy | AUC | Recall | Precision | F1-sore | cient |
| CatBoost | 0.7973 | 0.8851 | 0.7657 | 0.8266 | 0.7949 | 0.5952 |
| Light GBM | 0.7917 | 0.8773 | 0.7633 | 0.8186 | 0.7900 | 0.5840 |
| XGBoosting | 0.7714 | 0.8586 | 0.7336 | 0.8038 | 0.7671 | 0.5436 |
| Gradient | 0.7692 | 0.8577 | 0.7234 | 0.8067 | 0.7627 | 0.5393 |
| Boosting | | | | | | |
| Extra Trees | 0.7566 | 0.8317 | 0.7173 | 0.7892 | 0.7515 | 0.514 |
| Logistic | 0.7900 | 0.8481 | 0.7732 | 0.8090 | 0.7906 | 0.5802 |
| Regression | | | | | | |
| Random | 0.7268 | 0.7985 | 0.6521 | 0.7795 | 0.7101 | 0.4556 |
| Forest | | | | | | |
| Ada Boost | 0.7284 | 0.8073 | 0.7200 | 0.7430 | 0.7312 | 0.4568 |
| Decision | 0.6731 | 0.6743 | 0.6828 | 0.681 | 0.6818 | 0.3457 |
| Tree | | | | | | |
| Linear | 0.7203 | 0.7466 | 0.7292 | 0.7268 | 0.7280 | 0.4402 |
| Discriminant | | | | | | |
| Analysis | | | | | | |
| K Neighbors | 0.6127 | 0.6492 | 0.6364 | 0.6195 | 0.6278 | 0.2243 |
| Naive | 0.5422 | 0.5351 | 0.8496 | 0.5339 | 0.6556 | 0.0689 |
| Bayes | | | | | | |
| Quadratic | 0.5259 | 0.5251 | 0.5597 | 0.5660 | 0.4511 | 0.0505 |
| Discriminant | | | | | | |
| Analysis | | | | | | |

TABLE 4

| | | | | | | Kappa |
|---|---|---|---|---|---|---|
| Algorithm | | | | | | coeffi- |
| classifier | Accuracy | AUC | Recall | Precision | F1-sore | cient |
| CatBoost | 0.7945 | 0.8814 | 0.7595 | 0.8262 | 0.7912 | 0.5897 |
| Light GBM | 0.7921 | 0.8785 | 0.7650 | 0.8181 | 0.7905 | 0.5846 |
| XGBoosting | 0.7683 | 0.8552 | 0.7241 | 0.8047 | 0.7621 | 0.5375 |
| Gradient | 0.7665 | 0.8536 | 0.7237 | 0.8021 | 0.7607 | 0.5340 |
| Boosting | | | | | | |
| Extra Trees | 0.7599 | 0.8360 | 0.7234 | 0.7911 | 0.7556 | 0.5205 |
| Logistic | 0.7898 | 0.8505 | 0.7688 | 0.8117 | 0.7895 | 0.5799 |
| Regression | | | | | | |
| Random | 0.7088 | 0.7825 | 0.6293 | 0.7624 | 0.6892 | 0.4199 |
| Forest | | | | | | |
| Ada Boost | 0.7254 | 0.8034 | 0.7166 | 0.7400 | 0.7281 | 0.4509 |
| Decision | 0.6734 | 0.6749 | 0.6944 | 0.6776 | 0.6858 | 0.3460 |
| Tree | | | | | | |
| Classifier | | | | | | |
| Linear | 0.7240 | 0.7523 | 0.7374 | 0.7286 | 0.7327 | 0.4475 |
| Discriminant | | | | | | |
| Analysis | | | | | | |
| K Neighbors | 0.6097 | 0.6478 | 0.6395 | 0.6152 | 0.6271 | 0.2181 |
| Naive | 0.5439 | 0.5355 | 0.8418 | 0.5356 | 0.6545 | 0.0729 |
| Bayes | | | | | | |
| Quadratic | 0.5135 | 0.5181 | 0.3407 | 0.6133 | 0.3076 | 0.0361 |
| Discriminant | | | | | | |
| Analysis | | | | | | |

As shown in Table 2 to Table 4, when the antibiotic resistance assessing classifiers of the present disclosure are boosting algorithm classifiers, the accuracy to analyze the reference mass spectrum data of the drug-resistance database can reach more than 75%, and the area under the receiver operating characteristic curve (AUC) also can reach more than 85%. Thus, it shows that antibiotic resistance assessing classifier of the present disclosure and the drug-resistant microorganism assessing system can be used to assess whether the test microorganism is a drug-resistant microorganism or not effectively and has the potential application in relevant markets.

Further, please refer to Table 5, which shows the F1 scores of different combinations of the data interval value and 10 algorithm classifiers, namely LightGBM algorithm classifier, Gradient Boosting algorithm classifier, Logistic Regression algorithm classifier, XGBoost algorithm classifier, Extra Trees algorithm classifier, Random Forest algorithm classifier, Linear SVM algorithm classifier, Decision Tree algorithm classifier, K Neighbors algorithm classifier and Naive Bayes algorithm classifier (5-fold cross-validation, respectively), and the best combination of the data interval value and the algorithm classifiers is selected based on the following F1 score calculated by the following Formula (II). Formula (II) is shown below:

$$F 1 score = \frac{2 True positive}{2 True positive + False positive + False negative} . \qquad Formula ( II )$$

TABLE 5

| | Data Interval Value (Da) | | | | |
|---|---|---|---|---|---|
| Algorithm classifier | 1 | 5 | 10 | 15 | 20 |
| LightGBM | 0.8131 | 0.8121 | 0.8134 | 0.8086 | 0.8026 |
| Gradient Boosting | 0.7985 | 0.7962 | 0.8017 | 0.7952 | 0.7899 |
| Logistic Regression | 0.8036 | 0.7971 | 0.7973 | 0.7866 | 0.7801 |
| XGBoosting | 0.7943 | 0.8045 | 0.8020 | 0.7916 | 0.7900 |
| Extra Trees | 0.7688 | 0.7648 | 0.7702 | 0.7634 | 0.7700 |
| Random Forest | 0.7629 | 0.7462 | 0.7583 | 0.7589 | 0.7596 |
| Linear SVM | 0.7728 | 0.7891 | 0.7790 | 0.7568 | 0.7504 |
| Decision Tree | 0.7294 | 0.7264 | 0.7219 | 0.7209 | 0.7214 |
| K Neighbors | 0.6802 | 0.7001 | 0.7164 | 0.7009 | 0.7259 |
| Naive Bayes | 0.6775 | 0.6793 | 0.6770 | 0.6782 | 0.6783 |

As shown in Table 5, when the data interval value of the converted mass spectrum data is 1, 5 and 10 daltons, the F1 score of different algorithm classifiers can be up to 0.67, wherein the combination of 10 daltons and LightGBM algorithm classifier has the highest performance in the drug-resistance database of the present disclosure.

2. Analyzing the Assessing Accuracy of the Antibiotic Resistance Assessing Classifier of the Present Disclosure to Different Drug-Resistant Microorganisms

In the present test, the reference mass spectrum data in the drug-resistance database are trained by the antibiotic resistance assessing classifier of the present disclosure so as to analyze the assessing situation of the antibiotic resistance assessing classifier of the present disclosure to different drug-resistant microorganisms.

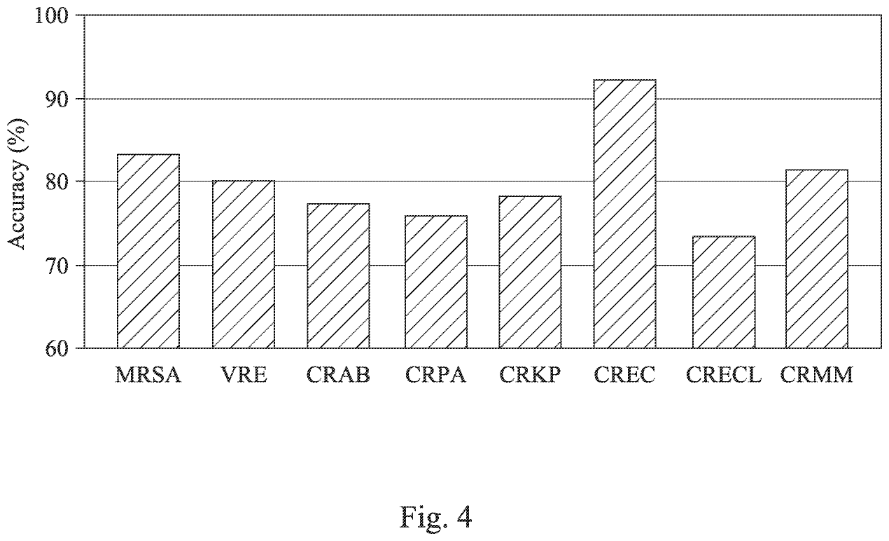

Please refer to FIG. 4, which shows assessed results of drug-resistant microorganisms obtained by the antibiotic resistance assessing classifier of the present disclosure that is used to analyze the conventional mass spectrometry data of different drug resistance microorganisms. As shown in FIG. 4, when the conventional mass spectrometry data of different drug-resistant microorganisms are trained by the antibiotic resistance assessing classifier of the present disclosure, all of the assessing accuracies of different drug-resistant microorganisms can reach more than 70%. It shows that the antibiotic resistance assessing classifier of the present disclosure can be used to analyze the mass spectrum data of the drug-resistant microorganisms commonly seen in clinical practice effectively and then output the assessed result of drug-resistant microorganism with high accuracy. Therefore, the method for assessing drug-resistant microorganism and the drug-resistant microorganism assessing system of the present disclosure have the potential to analyze the mass spectrum data obtained after the processing of the rapid sample processing method and has the potential application in relevant arts.

3. Analyzing the Best Range of the Mass-to-Charge Ratio of the Normalized Target Mass Spectrum Data in the Method for Assessing Drug-Resistant Microorganism and the Drug-Resistant Microorganism Assessing System of the Present Disclosure

In the present test, the reference mass spectrum data are trained by the drug-resistant microorganism assessing system according to the method for assessing drug-resistant microorganism of the present disclosure so as to confirm the best range of the mass-to-charge ratio of the normalized target mass spectrum data. In the experiment, the sliding window algorithm is used to analyze the accuracy of the assessed result of drug-resistant microorganism obtained from the reference mass spectrum data, which is trained by the method for assessing drug-resistant microorganism of the present disclosure, in the drug-resistance database. In particular, the sliding window algorithm sets a sliding window range of the accuracy of the assessed result of drug-resistant microorganism and the corresponding mass-to-charge ratio values, and the results of AUC are calculated in the current sliding window within the selected range of mass-to-charge ratio value by the sliding window every time, wherein the slide and the calculation are made in a frequency in an increment of 100 daltons every time until the mass-to-charge ratio of each of the reference mass spectrum data reaches the maximum (20,000 daltons) thereof.

The present test is performed based on Example 1 to Example 10, and the sliding window ranges of Example 1 to Example 10 are shown in Table 6.

TABLE 6

| | Sliding window range (Da) |
|---|---|
| Example 1 | 1,000 |
| Example 2 | 2,000 |
| Example 3 | 3,000 |
| Example 4 | 4,000 |
| Example 5 | 5,000 |
| Example 6 | 6,000 |
| Example 7 | 7,000 |
| Example 8 | 8,000 |
| Example 9 | 9,000 |
| Example 10 | 10,000 |

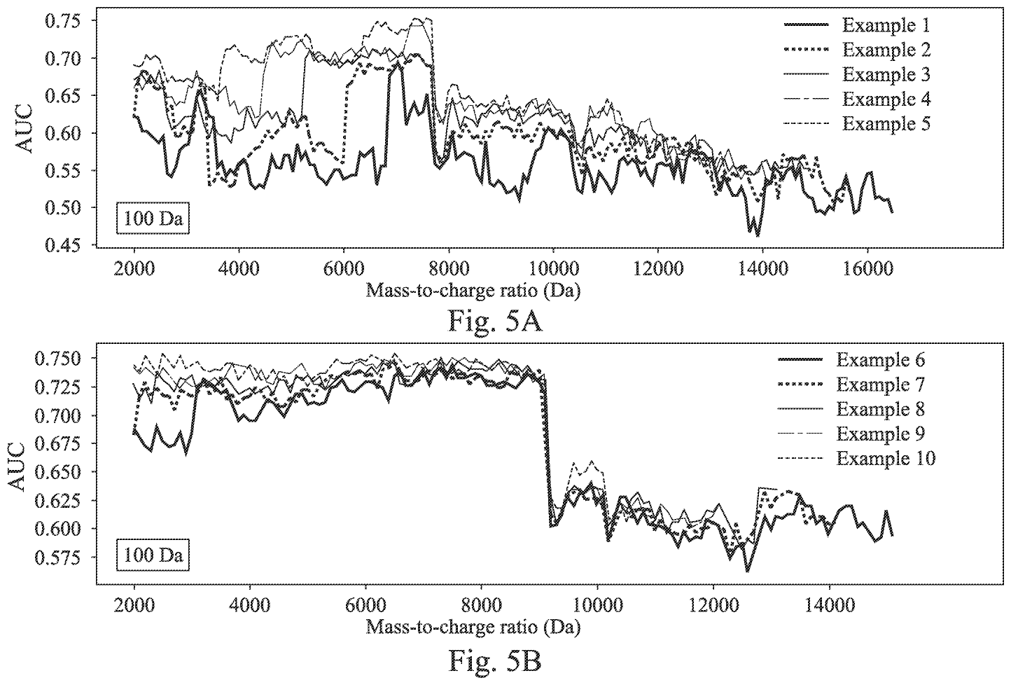

Please refer to FIG. 5A and FIG. 5B. FIG. 5A shows analyzed results of the sliding window algorithm of the methods for assessing drug-resistant microorganism of Example 1 to Example 5, wherein “100 Da” represents that the slide and the calculation are made in a frequency in an increment of 100 daltons every time. FIG. 5B shows analyzed results of the sliding window algorithm of the methods for assessing drug-resistant microorganism of Example 6 to Example 10, wherein “100 Da” represents that the slide and the calculation are made in a frequency in an increment of 100 daltons every time. As shown in FIG. 5A, when the sliding window range is between 1,000 daltons to 5,000 daltons, the assessing results of AUC of Example 1 to Example 5 have larger vibration amplitude. As shown in FIG. 5B, when the sliding window range is larger than 6,000 daltons, the assessing results of AUC that the mass-to-charge ratio ranging from 2,000 to 14,000 daltons are significantly enhanced, wherein the best range of the mass-to-charge ratio is between 4,000 to 12,000 daltons. Therefore, it shows that by selecting the normalized target mass spectrum data with a mass-to-charge ratio ranging from 2,000 to 14,000 daltons, the method for assessing drug-resistant microorganism and the drug-resistant microorganism assessing system of the present disclosure can be used to assess whether the test microorganism is a drug-resistant microorganism or not effectively and has the potential application in relevant markets.

4. The Method for Assessing Drug-Resistant Microorganism and the Drug-Resistant Microorganism Assessing System of the Present Disclosure are Used to Analyze the Mass Spectrum Data of the Samples Processed by the Rapid Sample Processing Method and the Conventional Sample Processing Method

In the present test, the samples infected by Staphylococcus aureus and the samples infected by Acinetobacter baumannii are respectively processed by the commercial kit (MBT Sepsityper® IVD kit) based on the user manual thereof, and the processed samples obtained therefrom are detected by MALDI-TOF method so as to obtain the corresponding mass spectrum data (“rapid mass spectrometry data” hereafter). At the same time, the samples infected by Staphylococcus aureus and the samples infected by Acinetobacter baumannii are respectively processed by the conventional sample processing method as aforementioned described, and the processed samples obtained therefrom are detected by MALDI-TOF method so as to obtain the corresponding mass spectrum data (“conventional mass spectrometry data” hereafter) for the subsequent analysis.

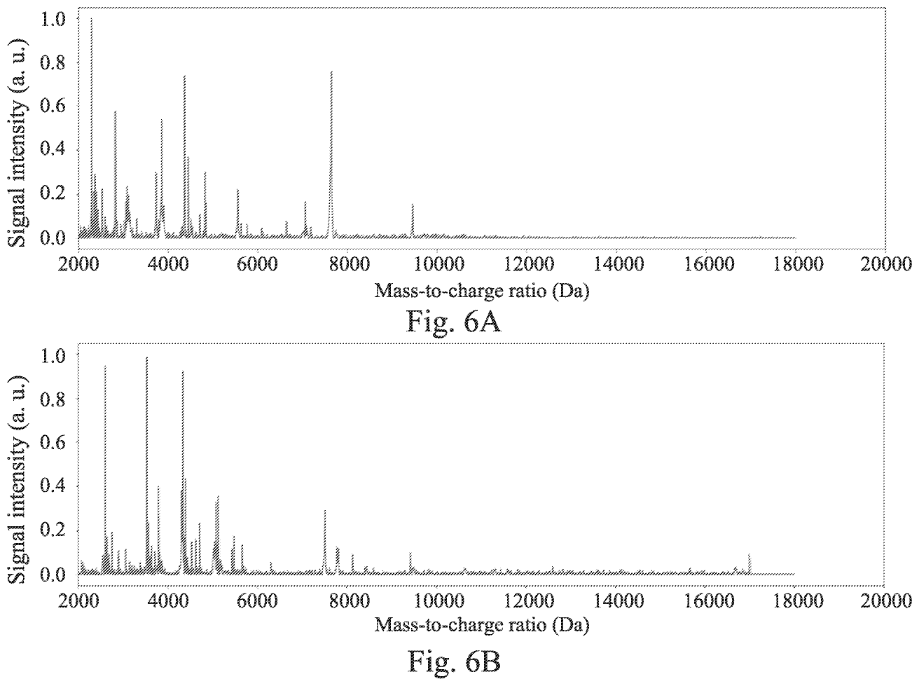

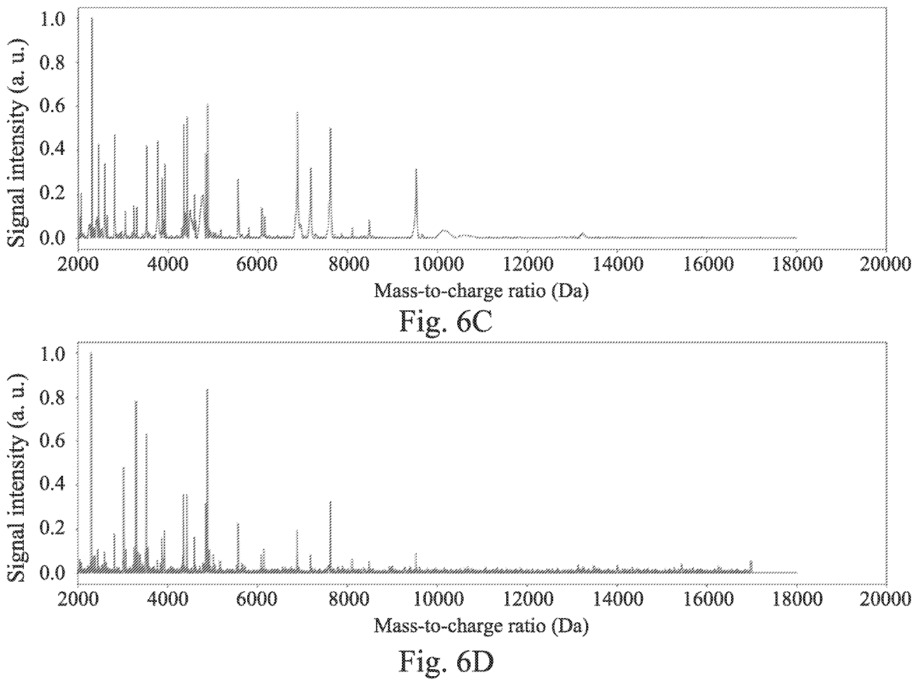

Please refer to FIG. 6A, FIG. 6B, FIG. 6C and FIG. 6D. FIG. 6A is a rapid mass spectrometry data of the sample infected with Acinetobacter baumannii. FIG. 6B is a conventional mass spectrometry data of the sample infected with Acinetobacter baumannii. FIG. 6C is a rapid mass spectrometry data of the sample infected with Staphylococcus aureus. FIG. 6D is a conventional mass spectrometry data of the sample infected with Staphylococcus aureus. As shown in FIG. 6A to FIG. 6D, in the rapid mass spectrometry data and the conventional mass spectrometry data of the sample infected by Acinetobacter baumannii as well as the rapid mass spectrometry data and the conventional mass spectrometry data of the sample infected by Staphylococcus aureus, the peak distribution range and the distributing trend thereof have significant differences, and the rapid mass spectrometry data and the conventional mass spectrometry data of different bacteria are also different. However, after processing by the sample pre-processing step of the present disclosure, the AUC thereof can reach more than 0.8. Thus, it shows that the method for assessing drug-resistant microorganism and the drug-resistant microorganism assessing system of the present disclosure can be used to analyze and train the conventional mass spectrometry data obtained from conventional sample processing method and the rapid mass spectrometry data obtained from rapid sample processing method simultaneously so as to output an accurate assessed result of drug-resistant microorganism. Therefore, the method for assessing drug-resistant microorganism and the drug-resistant microorganism assessing system of the present disclosure not only can effectively reduce the time required for conventional microbial culture, identification and antibiotic susceptibility testing so as to provide a more reliable basis for the use of antibiotics, and the harm caused by the complications of microbial infections can also be reduced.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A method for assessing drug-resistant microorganism, comprising:
performing a model establishing step, comprising:
providing a drug-resistance database, wherein the drug-resistance database comprises a plurality of reference mass spectrum data, and the reference mass spectrum data are obtained by processing a processed reference sample with a conventional sample processing method or a rapid sample processing method;
performing a reference spectrum pre-processing step, wherein the reference mass spectrum data are pre-processed so as to obtain a plurality of normalized reference mass spectrum data; and
performing a model training step, wherein the normalized reference mass spectrum data are trained to achieve a convergence by an algorithm classifier so as to obtain an antibiotic resistance assessing classifier;
providing a test sample, wherein the test sample comprises a test microorganism;
performing a sample pre-processing step, wherein the test sample is processed by the conventional sample processing method or the rapid sample processing method so as to obtain a processed sample;
performing an analysis step, wherein the processed sample is detected by a mass spectrometry method so as to obtain a target mass spectrum data;
performing a spectrum pre-processing step, wherein the target mass spectrum data is pre-processed so as to obtain a normalized target mass spectrum data;
performing a feature extraction step, wherein the normalized target mass spectrum data is trained to achieve a convergence by the antibiotic resistance assessing classifier so as to obtain a spectrum feature; and
performing an assessing step, wherein the spectrum feature is analyzed by the antibiotic resistance assessing classifier so as to output an assessed result of drug-resistant microorganism, and the assessed result of drug-resistant microorganism is for assessing whether the test microorganism is a drug-resistant microorganism or not.

2. The method for assessing drug-resistant microorganism of claim 1, wherein the test sample is processed by a step-by-step centrifuging method in the rapid sample processing method, and the step-by-step centrifuging method comprises:
performing a centrifuging step, wherein the test sample is processed by a plurality of centrifugations so as to obtain a centrifuged sample, and the centrifuged sample comprises the test microorganism;
performing a reactive step, wherein a reaction reagent is added to the centrifuged sample and then well mixed so as to obtain a post-reaction sample; and
performing a final centrifuging step, wherein the post-reaction sample is centrifuged so as to obtain the processed sample;
wherein the reaction reagent comprises thioglycolate broth, ethanol, formic acid or acetonitrile.

3. The method for assessing drug-resistant microorganism of claim 1, wherein the spectrum pre-processing step comprises:
performing a calibration step, wherein a background noise of the target mass spectrum data is removed so as to obtain a first processed target mass spectrum data;
performing a sampling normalization step, wherein a temporal resolution value of the first processed target mass spectrum data is adjusted so as to obtain a second processed target mass spectrum data;
performing a spectrum conversion step, wherein the second processed target mass spectrum data is processed by a mass-to-charge ratio conversing method so as to obtain a converted mass spectrum data; and
performing a binning step, wherein a data interval value of the converted mass spectrum data is adjusted so as to obtain the normalized target mass spectrum data.

4. The method for assessing drug-resistant microorganism of claim 3, wherein a mass-to-charge ratio of the normalized target mass spectrum data ranges from 2,000 to 14,000 daltons.

5. The method for assessing drug-resistant microorganism of claim 4, wherein the mass-to-charge ratio of the normalized target mass spectrum data ranges from 4,000 to 12,000 daltons.

6. The method for assessing drug-resistant microorganism of claim 1, wherein the drug-resistant microorganism is Methicillin-resistant Staphylococcus aureus (MRSA), Vancomycin-resistant Enterococci (VRE), Carbapenem-resistant Acinetobacter baumannii (CRAB), Carbapenem-resistant Pseudomonas aeruginosa (CRPA), Carbapenem-resistant Klebsiella pneumoniae (CRKP), Carbapenem-resistant Escherichia coli (CREC), Carbapenem-resistant Escherichia cloacae (CRECL), or Carbapenem-resistant Morganella morganii (CRMM).

7. The method for assessing drug-resistant microorganism of claim 1, wherein the mass spectrometry method is MALDI-TOF (matrix assisted laser desorption ionization time-of-flight) method.

8. The method for assessing drug-resistant microorganism of claim 1, wherein the reference spectrum pre-processing step comprises:
performing a reference calibration step, wherein a background noise of each of the reference mass spectrum data is removed so as to obtain a plurality of first processed reference mass spectrum data;
performing a reference sampling normalization step, wherein a temporal resolution value of each of the first processed reference mass spectrum data is adjusted so as to obtain a plurality of second processed reference mass spectrum data;
performing a reference spectrum conversion step, wherein each of the second processed reference mass spectrum data is processed by a mass-to-charge ratio conversing method so as to obtain a plurality of converted reference mass spectrum data; and
performing a reference binning step, wherein a reference data interval value of each of the converted reference mass spectrum data is adjusted so as to obtain the normalized reference mass spectrum data.

9. The method for assessing drug-resistant microorganism of claim 1, wherein the algorithm classifier is a boosting algorithm classifier.

10. The method for assessing drug-resistant microorganism of claim 1, wherein a mass-to-charge ratio of each of the normalized reference mass spectrum data ranges from 2,000 to 14,000 daltons.

11. A drug-resistant microorganism assessing system, comprising:
a non-transitory machine readable medium for storing a target mass spectrum data, wherein the target mass spectrum data is obtained by detecting a processed sample by a mass spectrometry method, the processed sample comprises a test microorganism, and the processed sample is obtained by a conventional sample processing method or a rapid sample processing method; and
a processor signally connected to the non-transitory machine readable medium, wherein the processor comprises a drug-resistant microorganism assessing program, and the drug-resistant microorganism assessing program comprises:
a spectrum pre-processing module for pre-processing the target mass spectrum data so as to obtain a normalized target mass spectrum data, wherein the spectrum pre-processing module comprises:
a calibration unit for removing a background noise of the target mass spectrum data so as to obtain a first processed target mass spectrum data;
a sampling normalization unit signally connected to the calibration unit, wherein the sampling normalization unit is for adjusting a temporal resolution value of the first processed target mass spectrum data so as to obtain a second processed target mass spectrum data; and
a spectrum conversion unit signally connected to the sampling normalization unit, wherein the spectrum conversion unit is for processing the second processed target mass spectrum data by a mass-to-charge ratio conversing method so as to obtain a converted mass spectrum data, and then a data interval value of the converted mass spectrum data is adjusted by the spectrum conversion unit so as to obtain the normalized target mass spectrum data; and
an antibiotic resistance assessing classifier signally connected to the spectrum pre-processing module, wherein the normalized target mass spectrum data is trained to achieve a convergence by the antibiotic resistance assessing classifier so as to obtain a spectrum feature, and the spectrum feature is analyzed by the antibiotic resistance assessing classifier so as to output an assessed result of drug-resistant microorganism;
wherein the antibiotic resistance assessing classifier is established by a model establishing step, and the model establishing step comprises:
providing a drug-resistance database, wherein the drug-resistance database comprises a plurality of reference mass spectrum data, and the reference mass spectrum data are obtained by processing a processed reference sample with a conventional sample processing method or a rapid sample processing method;
performing a reference spectrum pre-processing step, wherein the reference mass spectrum data are pre-processed so as to obtain a plurality of normalized reference mass spectrum data; and
performing a model training step, wherein the normalized reference mass spectrum data are trained to achieve a convergence by an algorithm classifier so as to obtain the antibiotic resistance assessing classifier;
wherein the assessed result of drug-resistant microorganism is for assessing whether the test microorganism is a drug-resistant microorganism or not.

12. The drug-resistant microorganism assessing system of claim 11, wherein the mass spectrometry method is MALDI-TOF method.

13. The drug-resistant microorganism assessing system of claim 11, wherein a mass-to-charge ratio of the normalized target mass spectrum data ranges from 2,000 to 14,000 daltons.
